(19) United States
(12) Patent Application Publication
(10) Pub. No.: US 2011/0230793 A1
(43) Pub. Date: Sep. 22, 2011

(54) Treatment of Alopecia with ultrasound

(75) Inventors: Eugene A. Larson, Lummi Island, WA (US); Perry W. Kaminski, Mukilteo, WA (US)

(73) Assignee: SONORHYTHM LLC

(21) Appl. No.: 12/931,941

(22) Filed: Feb. 15, 2011

Related U.S. Application Data
(60) Provisional application No. 61/340,359, filed on Mar. 16, 2010.

(51) Int. Cl. A61N 7/00

(52) U.S. Cl. 601/2

(57) ABSTRACT

A method for the treatment of alopecia and in particular the problem of male-pattern baldness. By application of controlled ultrasound exposures to specific regions in the skin tissue, a physiological response will develop which causes cells in the hair follicle to increase in number, and/or new follicles to form or multiply in response to controlled damage to existing cells, leading to new hair follicles and/or increased shaft thickness in existing hair follicles resulting in more robust hair growth.

This application claims the benefit of U.S. Provisional Patent Application No. 61/340,359 filed Mar. 16, 2010, the entire disclosure of which is hereby incorporated by reference.

### FIELD OF THE INVENTION

The present invention is directed to a method and apparatus to stimulate hair follicle rejuvenation in people who have experienced Alopecia, especially male-pattern baldness. In particular, the invention utilizes high-intensity ultrasound, applied to the subcutaneous tissue in the vicinity of existing hair follicles, which results in one or more of the following: cell growth, hair follicle rejuvenation, invigorated hair follicles, increased hair-shaft size, and new hair growth.

### BACKGROUND OF THE INVENTION

Alopecia is the medical description for loss of hair from the head or the body. Androgenetic alopecia (male- or female-pattern hair loss) is usually an unwelcomed event with social and psychological consequences. In addition to a receding hairline, a bald area often develops on the vertex of the scalp, thought to be triggered by dihydrotestosterone (DHT), a powerful sex hormone. This specific baldness pattern is often referred to as Androgenetic alopecia. Alopecia affects both sexes and by age 80 significant head alopecia (mid-frontal hair loss) affects over 50% of women and 70% of men (FIG. 1). Of particular concern to men is male-pattern baldness, which affects over 40 million men in the United States, of which 25% begin balding by age 30 and ⅔ by age 60. Male pattern baldness is characterized by receding hairlines, which can be noticeable beginning in the late teens. Scarring alopecia, on the other hand, is the result of complete destruction of the hair follicle which cannot be rejuvenated and is not the subject of the present invention.

In the male pattern baldness syndrome, the hair follicle diminishes in overall size and most important, in diameter. The resulting hair shaft width decreases until the scalp hair is very fragile and breaks off soon after being emitted from the scalp (FIG. 2). The hair follicle remains in a dormant state. Research has indicated that tissue removed from the scalp of men with alopecia grew hair when transplanted into the certain strains of laboratory mice, indicating that the hair follicle, while miniaturized and dormant, remains viable in male-pattern baldness.

Many pharmacological agents have been utilized to retard or reverse male pattern alopecia. One proposed method it to apply a polypeptide hair growth factor isolated from adipocytes and applied topically. Those approved for the treatment of alopecia by the FDA include Finasteride, an antiandrogen that inhibits the enzyme that converts testosterone to dihydrotestosterone (DHT) and Minoxidil, a vasodilator and potassium channel agonist. With pharmacological therapy, when the therapy is terminated the hair alopecia process of hair thinning and baldness resumes.

While previous therapies for alopecia have been directed at either interrupting the immune or androgen response or in stimulating angiogenesis and blood flow, ultrasound therapy alone has not been applied to scalp or skin tissue for stimulating new growth of cells in the hair follicle by cells in and/or around the hair follicle, or to the formation of new follicles.

The above is evident, for example, from US 2007/0016117 A1 to Sliwa, Jr. et al. which teaches invigorating hair growth by delivering acoustic energy, at low power levels, through hair shafts to the hair roots such that acoustic treatment is delivered directly to the hair roots and not to scalp tissues between such root regions. It is an aim of the process to not treat the scalp or expose the scalp to any treatment energy and to avoid the transmission of energy into scalp tissues.

In another method disclosed by US 2007/0078290 A1 to Esenaliev, growth of hair follicles is stimulated by the application of cavitation-enhancing nano-particles or micro-particles to the skin followed by irradiating that part of the skin with ultrasonic energy. Additionally, one or more hair growth promoting agents may be utilized in combination with the nano-particles or micro-particles.

Research has indicated that sub-lethal injury to cardiac muscle tissue, as well as other tissues induces repair and remodeling of tissue combined with the formation of blood vessels (angiogenesis). Various methods of experimental introduction of energy to injure and/or stimulate cardiac muscle tissue include laser tissue perforation and acoustic shock waves.

Recent studies have demonstrated that the application of extracorporeal shock waves to a variety of tissues can cause cell injury thereby stimulating a repair mechanism that may involve VEGF (Vascular Endothelium Growth Factor) upregulation, neovascularization (angiogenesis), NO (nitric oxide) synthesis, reduced ischemic necrosis, enhanced blood perfusion, increased permeability of cell membranes enhancing the release of growth factors, and other cell injury mechanisms that stimulate new tissue regeneration.

The physiological mechanism(s) through which this multitude of positive responses is generated is most likely the result of lethal and/or sublethal damage to the cellular structure in and near the region of interest. In many of these experiments that have demonstrated these positive effects of shock waves, the shock wave amplitudes (positive and negative pressure amplitudes) are sufficient to generate acoustic cavitation (the growth and subsequent violent collapse of bubbles). It is known that acoustic cavitation can result in extreme local energy concentrations sufficient to comminute kidney stones. Current approaches for inducing cell injury and tissue repair often utilize high amplitude shock waves to stimulate this repair. However, it is also known that low energy pulsed ultrasound can also stimulate wound healing and tissue repair. Although the exact causes of these stimulate repair mechanisms is unclear, it is widely understood that inducing some lethal or sublethal damage to the tissue somehow stimulates the body to physiologically respond to this stimulus by up-regulating the body's own internal repair mechanisms.

### SUMMARY OF THE INVENTION

The present invention sets forth a method and apparatus for the treatment of alopecia and in particular the problem of male-pattern baldness. By application of controlled ultrasound exposures to specific regions in the skin tissue, without the introduction of external agents (such as nanoparticles or pharmacological agents) a physiological response will develop which causes cells in the hair follicle, and/or new follicles, to form or multiply in response to controlled damage to existing cells, leading to new and/or increased shaft thickness and more robust hair growth. These rejuvenated hair follicles support increased thickness and density of hair.

### BRIEF DESCRIPTION OF THE DRAWINGS

FIG. 1 illustrates male and female hair loss patterns showing progressive degrees of Alopecia in both men and women, especially male-pattern baldness.

FIG. 2 shows normal hair growth phases comprising (1) anagen, (2) catagen and (3) telogen.

FIG. 3 illustrates a scalp layer showing typical sparse hair pattern of alopecia.

FIG. 4 illustrates insonification of a hair follicle with focused wave ultrasound applied by a combination imaging and high intensity ultrasound applicator.

FIG. 5 shows insonification of alopecia region with plane or focused wave ultrasound

### DETAILED DESCRIPTION OF THE PREFERRED EMBODIMENT

The details of the present invention will now be discussed with reference to the drawings which represent the invention by way of example only.

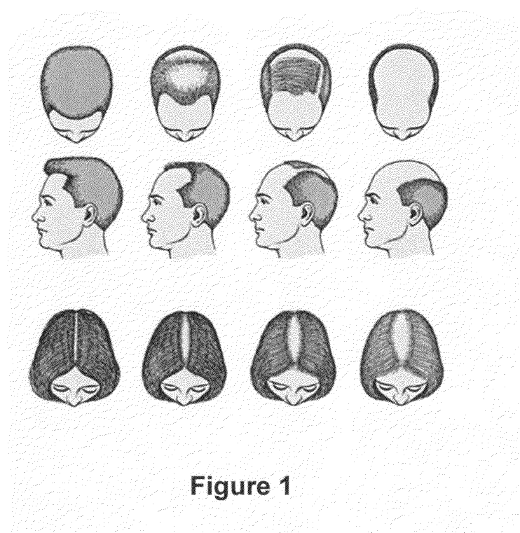

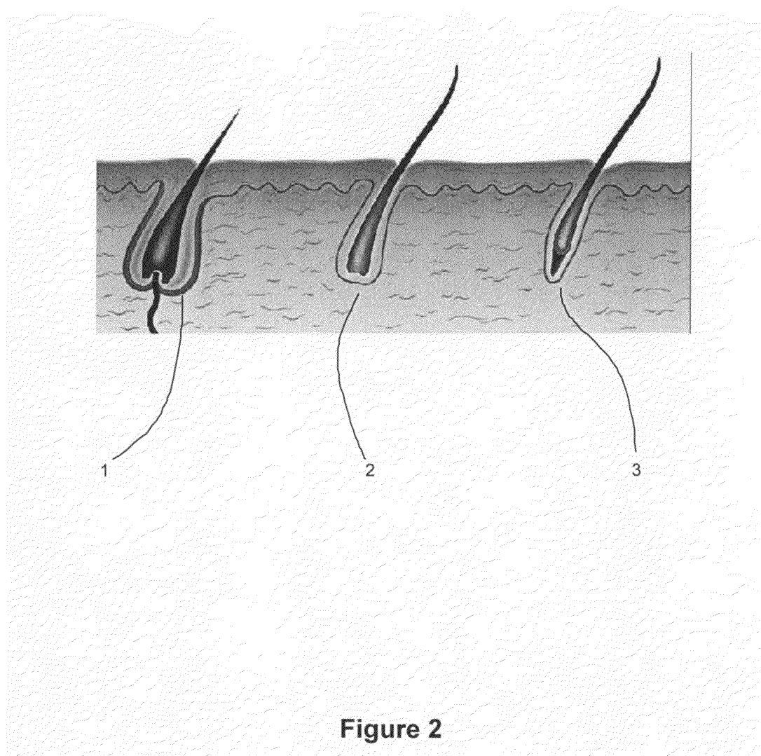

FIG. 1 shows progressive degrees of alopecia in both men and women, especially male-pattern baldness. FIG. 2 illustrates normal hair growth phases, namely:
Anagen 1—active growth phase which lasts up to several years. At any given time, the majority (85%) of human body hair is in this phase. During anagen, the hair has an abundance of melanin.
Catagen 2—regressive phase, which lasts about two weeks, during which the hair stops growing but is not yet shed. About 3-4% of human body hair is in this phase at any given time.
Telogen 3—resting phase (telogen phase), which lasts 5-6 weeks, at the end of which the hair falls out and a new hair begins to form. Approximately 10-13% of human body hair is in this phase at any one time.

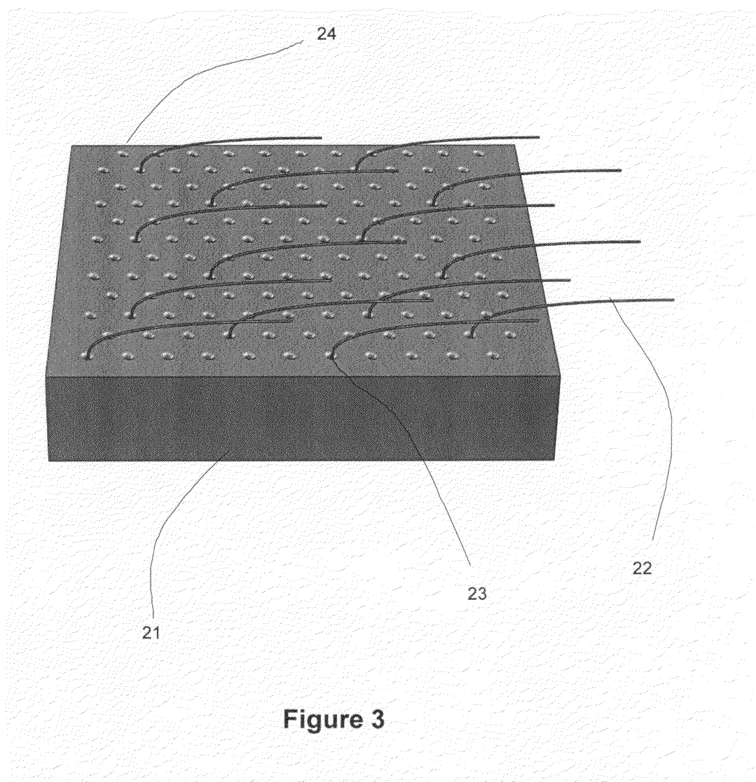

A scalp layer showing typical sparse hair pattern of alopecia is shown in FIG. 3 comprising the scalp layer 21, hair shaft 22, healthy hair follicle in anagen phase 23 and dormant hair follicle in telogen phase 24.

The present invention describes the application of high intensity ultrasound to stimulate the release of cellular growth factors, produce acoustic cavitation, locally-induced boiling, and/or locally confined high thermal dose to induce lethal and/or sub-lethal damage to selective cells within subcutaneous tissue, such as dormant hair follicles (and cells within their vicinity). This application of ultrasound results in an autologous physiological response to this stimulus, and resulting in the repair of damaged tissue, and the regeneration of new tissue, including hair follicle cells that would produce new (and or stronger) hair growth to treat the problem of male-pattern baldness and other susceptible forms of alopecia.

Some recent experiments have demonstrated that short High Intensity Ultrasound bursts can result in complete emulsification of cellular tissue within various animal tissues. When these high intensity ultrasound bursts are on the order of a few microseconds in length and at very large acoustic pressure amplitudes, violent acoustic cavitation activity is generated. This violent cavitation induces such strong mechanical stresses that cell structures are destroyed and a liquid emulsion results. When such very short high intensity ultrasound pulses of high amplitude are used, very little, if any, temperature rise will occur. This approach to tissue emulsification is called “histotripsy”. In the present invention, however, the inventors propose not destroying the entire hair follicle, but rather limiting the region of tissue emulsification to at least some cells in or around the hair follicle to stimulate the generation or release of growth factors which will rejuvenate the dormant hair follicle and/or cause new hair follicles to form from stimulated stem cells, initiating new hair follicles and/or increased hair shaft diameter in existing hair follicles resulting in more robust hair growth.

Other recent experiments have also demonstrated that when longer high intensity ultrasound pulses are used, but with amplitudes less than those used in histotripsy, localized boiling can be induced within a few milliseconds.

Because sound absorption in tissue is dependent on the acoustic frequency, higher frequencies cannot be propagated to large distances, but result in much stronger absorption, and thus in more rapid local temperature rises. An important aspect of high intensity ultrasound-induced boiling with short (e.g. millisecond-length) acoustic pulses is that the acoustic waveform must generate a shock wave so that the heating is generated by the broad harmonic spectrum residing within the shock wave itself. The process of generating localized boiling by millisecond length high intensity ultrasound pulses is called shockwave-induced boiling.

There are significant advantages to the use of shock-wave induced boiling to induce localized damage to subcutaneous tissue. Because the high-intensity ultrasound pulses need to propagate through a specific distance before it generates a shock, much less absorption is experienced by the ultrasound pulse until it reaches a critical well-defined distance. In shockwave-induced boiling, almost all the energy carried in the acoustic pulse is delivered only at the point of shock wave generation. This localized energy deposition at the point of shock formation is important to the present invention in that stimulation occurs only in the subcutaneous layer, and not in proximal, near-surface, layers. Furthermore, little if any of the ultrasound energy continues to propagate to the skull. In the present invention, the acoustic parameter space is chosen so that no significant energy deposition occurs outside the region of interest, and in particular, no significant trans-skull propagation occurs. This approach is in contrast with other methods that do not control the depth of penetration of the ultrasound-induced stimulus, and can result in surface damage to the skin or penetration of the high intensity ultrasound into the brain. Furthermore, it is superior to cavitation-induced damage as shockwave-induced boiling always occurs at the transducer focus as opposed to cavitation-induced damage which can occur pre-focally and sporadically as it requires a nucleation site.

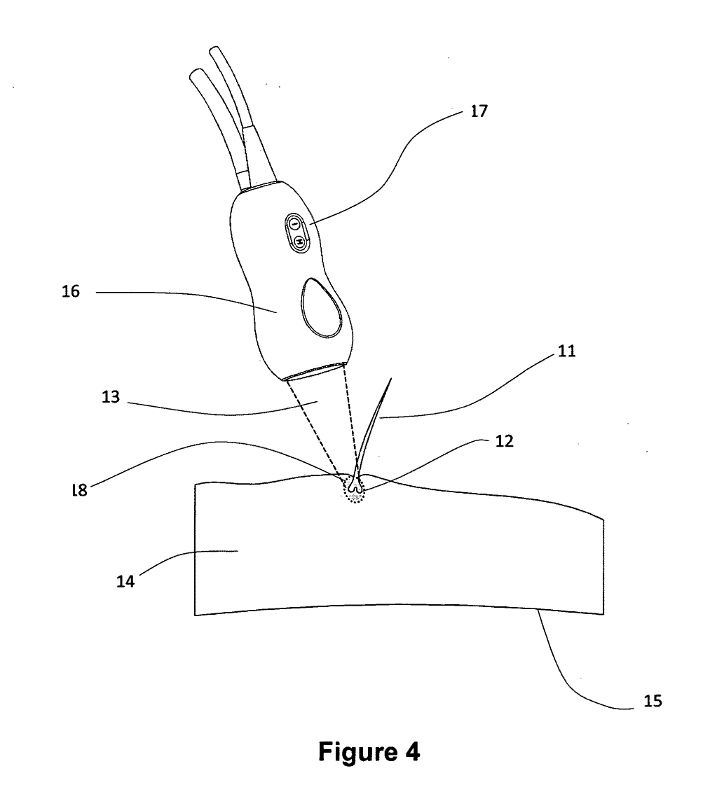

FIG. 4 illustrates an apparatus for stimulating cell growth by delivering high-intensity ultrasound to very precisely-located sites to induce acoustic cavitation, hyperthermia and/or localized boiling that will result in damage to cells of a specific and critical region in the skin tissue. In particular, FIG. 4 shows a hair shaft 11, hair follicle 12, subcutaneous tissue 14 and surface of the skull 15. A combination imaging and high intensity ultrasound probe is shown at 16 with an ultrasound beam 13 denoted by the dashed lines 13. The ultrasound probe 16 preferably includes a switch 17 to select “I” for imaging or “H” for high intensity ultrasound. The treatment zone is shown at 18. For the sake of viewing clarity, an acoustic coupling medium between the ultrasound probe 16 and the tissue 14 has been omitted.

The high intensity ultrasound applicator of FIG. 4 preferably produces lethal or non-lethal damage sites in the subcutaneous layer. The imaging transducer is preferably embedded within the high intensity ultrasound transducer and co-registered with this transducer so that image-guided placement of the high intensity ultrasound-induced lesions can be accomplished. An imaging transducer coaxially embedded within the high intensity ultrasound transducer (FIG. 4) would enable precise targeting of the subcutaneous layer.

Alternatively, the high intensity ultrasound (therapy) transducer may be composed of several individual transducer elements (such as an annular array) that would permit electronic targeting. Preferably, the multiple-element transducer array (either in an annular or 2D configuration) would have the capability of slight mechanical and/or electronic changes in the focus so that appropriate targeting could be achieved.

The high-intensity ultrasound delivered by the apparatus will result in lethal and or sublethal damage at the required site. Another aspect of the present invention is the acoustic waveforms that are used to induce the localized damage. These acoustic waveforms are preferably restricted to a narrow parameter space. Preferably, the applicator utilizes a transducer capable of delivering high intensity ultrasound pulses of varying pulse lengths ranging from microseconds to milliseconds, with acoustic intensities at the focus ranging from a few thousand to tens of thousands of W/cm2, and with acoustic frequencies ranging from a few MHz to approximately 20 MHz. The transducer is preferably driven by an electrical drive system consisting mainly of a frequency generator and a power amplifier that would drive the transducer at the required acoustic outputs.

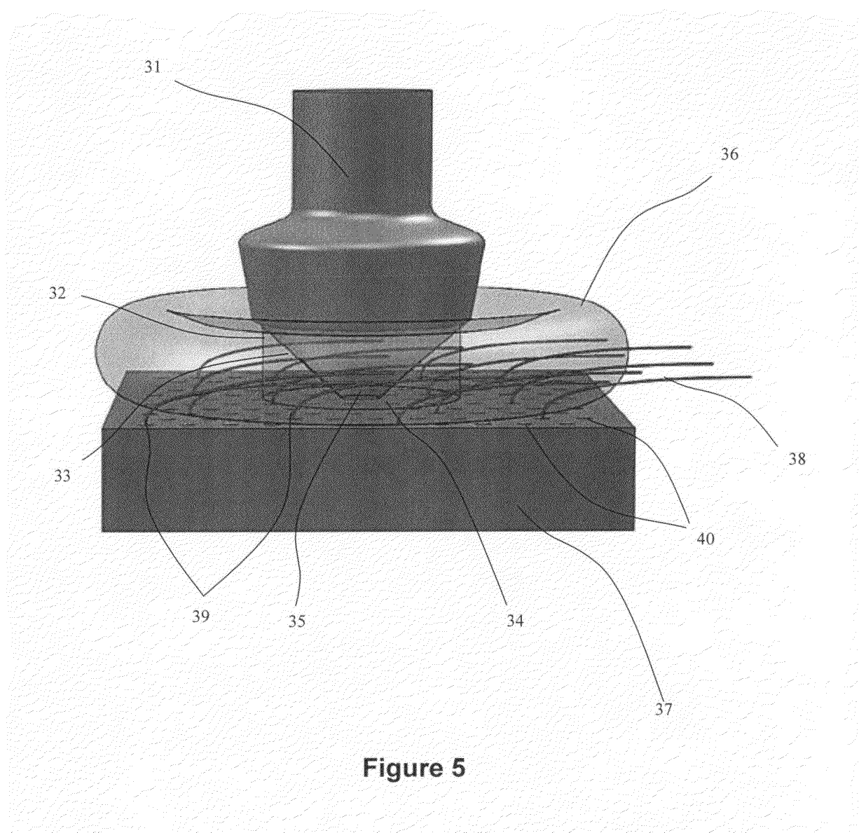

Another aspect of the present invention is that the high intensity ultrasound-generating applicator (e.g. FIG. 5) may include means to permit the surface of the skin to be cooled by a water (or other liquid) coupling bag, or by a gel or hydrogel that would be in touch with the patient's skin. Thus, a propagation medium exists that permits the high intensity ultrasound waveform to achieve its shock-propagation distance, as well as to cool the skin surface. The coupling means (e.g., water, gel or hydrogel) would prevent the surface of the transducer from exceeding its working temperature as well as cool the surface of the patient's skin.

FIG. 5 shows insonification of alopecia region with plane wave ultrasound or focused wave ultrasound by means of an ultrasound probe 31, plane wave ultrasound beam 32, focused wave ultrasound beam 33, plane wave insonification region 34, focused wave insonification region 35, gel, hydrogel or water bag coupling medium 36, scalp layer 37, hair shaft 38, healthy hair follicle in anagen phase 39 and dormant hair follicle in telogen phase 40. For focused wave, the transducer of FIG. 5 is preferably a single-element, curved transducer with an f-number close to 1.0, so that acoustic energy can be delivered to the desired position within the subcutaneous tissue. By inflating the water bag to various levels, or moving the transducer within a water bath, the high intensity ultrasound focus could then be shifted to accommodate the various conditions of individual patients.

For most patients, the thickness of the scalp skin would vary so little that little accommodation for different distances between the transducer and the desired location within the subcutaneous layer would be necessary. The high intensity ultrasound (therapy) transducer, therefore, may have a fixed focal length and placement of the lesion sites is done by pre-application measurements. However, if desired, several individual transducer elements (such as an annular array) that would permit electronic and/or mechanical targeting may be utilized. Alternatively, various thickness of coupling media may be used to vary the focal point of the transducer.

Another aspect of the present invention is that acoustic frequencies on the order of several megahertz can be utilized. Because sound absorption in water is relatively low with respect to tissue, acoustic frequencies as high at 20 MHz may be propagated through the water without major amplitude diminution, while such high frequencies would be rapidly absorbed in the tissue, and thus prevent further propagation through the patient's skull.

Utilizing a focused ultrasound transducer, the volume of the high intensity ultrasound focus, and especially the length of the focal envelop along the acoustic axis, which depends on the acoustic wavelength of the propagating waveform, will be very small if frequencies on the order of 10 MHz or above are used. For maximum effectiveness, it is preferable to deliver many small sites of damage rather than a few large ones, as the number of new and/or regenerated hair follicles will be correlated with the sites of cell stimulation.

For example, a typical procedure for the treating of a patient may be as follows:

1. The high intensity ultrasound applicator containing either transducer configuration (FIG. 4 or 5) would be connected to the electrical drive system.

2. In a preferred embodiment, which would include an imaging transducer embedded within the high intensity ultrasound therapy transducer (FIG. 4), the high intensity ultrasound applicator would be applied to the head of the patient, and the various skin layers would be imaged. With an imaging transducer working in the frequency range of 10-30 MHz, these various skin layers could be easily discerned.

3. Once the region of interest was targeted, either a short burst of high intensity ultrasound energy of (a) approximately 50 microseconds with an intensity of approximately 20 kW/cm², (to produce acoustic cavitation) or (b) approximately 20 milliseconds with an intensity of approximately 5 kW/cm2 would, be delivered (to produce localized boiling).

4. The focal region would be re-imaged to determine if hyperechoic spots were observed, as these spots are often associated with damage sites produced by high intensity ultrasound bursts.

5. After it was determined that a suitable damage site was induced, the transducer would be moved to a different location and the procedure repeated.

6. Multiple treatments may be required to achieve significant hair restoration.

While the invention has been described with reference to preferred embodiments it is to be understood that the invention is not limited to the particulars thereof. The present invention is intended to include modifications which would be apparent to those skilled in the art to which the subject matter pertains without deviating from the spirit and scope of the appended claims.

What is claimed is:

1. An ultrasound method for restoring and/or enhancing the presence of hair in a region of the skin undergoing alopecia, said method consisting of:
applying ultrasound to produce lethal and/or sub-lethal damage sites in a subcutaneous layer of a target tissue whereby upon subsequent physiological wound healing and repair of said damage sites, regenerated tissue will be formed,
said regenerated tissue including at least one of rejuvenated hair follicles and new hair follicles thereby resulting in the generation of at least one of new and thicker hair shafts, and a more robust growth of hair in said region of the skin undergoing alopecia.

2. The method of claim 1 wherein said ultrasound comprises High Intensity Ultrasound having an acoustic waveform whereby a shock wave is generated at said target tissue.

3. The method of claim 2 wherein tissue stimulation occurs only in said target tissue.

4. The method of claim 1 wherein said ultrasound further comprises imaging ultrasound.

5. The method of claim 1 including means to cool the skin in the area of the target tissue.

6. The method of claim 1 wherein said ultrasound is delivered via a plurality of individual transducer elements.
